(19) United States
(12) Patent Application Publication
(10) Pub. No.: US 2021/0054327 A1
(43) Pub. Date: Feb. 25, 2021

(54) Single Use Container Including a Collapsible Baffle Having Channels

(71) Applicant: EMD Millipore Corporation, Burlington, MA (US)

(72) Inventors: Jeffrey Pearsons, Burlington, MA (US); Amy Wood, Concord, MA (US); David Kraus, Amherst, NH (US)

(21) Appl. No.: 16/964,646

(22) PCT Filed: Mar. 13, 2019
(86) PCT No.: PCT/US2019/022026

§ 371 Date: Jul. 24, 2020

Related U.S. Application Data
(60) Provisional application No. 62/655,277, filed on Apr. 10, 2018.

(51) Int. Cl. C12M 1/00; C12M 3/00; C12M 1/06; C12M 1/34

(52) U.S. Cl. CPC C12M 27/20; C12M 23/14; C12M 23/26; C12M 41/42; C12M 29/14; C12M 27/02; C12M 23/48

(57) ABSTRACT

A collapsible container for a fluid that includes a flexible material, defining an internal working volume; at least one collapsible baffle adhered within the working volume of said collapsible container, the at least one baffle having one or more channels for delivering one or more fluids into the working volume via at least one hole in said one or more channels, one or more channels in said container for exiting or venting fluids from the working volume, and an impeller assembly disposed at least partially within said working volume of said container.

### REFERENCE TO RELATED APPLICATIONS

This present application claims the benefit of priority of U.S. Provisional Patent Application No. 62/655,277, filed Apr. 10, 2018, which is incorporated by reference herein in its entirety.

### BACKGROUND

### Field of the Invention

Embodiments of the present disclosure relate to a collapsible container useful as a mixer or a bioreactor. More particularly, some embodiments disclosed herein include a collapsible baffle(s) having channels and holes formed within an interior portion of the baffle(s) for the delivery of liquids and/or gases within an inner volume of the collapsible container.

### Description of the Related Art

Traditionally, fluids have been processed in systems that utilize stainless steel containers. These containers are sterilized after use so that they can be reused. The sterilization procedures are expensive and cumbersome as well as being ineffectual at times.

To provide greater flexibility in manufacturing and reduce times needed to sterilize and regenerate the equipment, manufacturers now utilize disposable sterilized containers, such as bags, that are used once for processing a product batch and are subsequently discarded. These disposable bags consist of a system for mixing two or more ingredients, at least one of which is liquid and the other(s) being liquid or solid, wherein the bag has a mixing element or the like for causing the contents to mix as uniformly as possible.

For example, a container, bag, bioreactor, or fermenter processes cells that are either in suspension or on microcarriers, wherein the bag further includes a circulating member such as an impeller for circulating and/or mixing liquid, gases, and, in some cases, the cells within the inner volume of the bag. Microcarriers are beads that comprise, for e.g., one or more of different materials, including various proteins, ceramics, and/or polymers, including DEAE-dextran, collagen, alginate, glass, polystyrene plastic, and acrylamide. Suitable commercial microcarriers include, but are not limited to, CYTODEX® microcarriers available from GE Corp.; SOLOHILL™ microcarriers available from Pall Corp., and CELLBIND® microcarriers available from Coming Inc

Some bags, bioreactors, or fermenters include a baffle formed vertically along at least a portion of an inner sidewall of the bag for improved mixing. These baffles are typically sleeves and often have a rigid member such as wood, plastic, or metal shaped to fit into an interior of the sleeves. The rigid member(s) are optionally inserted into the baffle for supporting the baffle and/or improving its mixing capability. Alternatively, the sleeve can be secured and stretched across portions of two inner, vertical surfaces of the container, such as one lower sidewall portion and an opposite upper sidewall portion to attain rigidity.

Large volume bags, e.g., 1000 L to 2000 L volume bags, containers, or bioreactors, present challenges for incorporating a rigid baffle, because the increased height of these systems makes it difficult to introduce the rigid insert into the baffle sleeve, presenting potential failure modes, tearing, abrasions, introduction of contaminants into the processed liquid, etc. In addition, the unfavorable bottom to top mixing evident in the smaller scaled bags becomes even more pronounced in larger bags because as the overall height of the bag increases, despite the reduced height to width aspect ratios, mixing efficiency decreases.

Also, as in the production of vaccines, the liquids involved often contain aluminum salt as an adjuvant, which improves the efficacy of the vaccine by enhancing the body's immune response. Unfortunately, the aluminum salts consist of particle sizes larger than 0.2 μm, and thus sterile filtering generally is not an option. As a result, minimizing the number of containers into which the vaccine needs to be transferred, since each transfer represents a potential breach of sterility, and the resulting contamination can't be filtered away, is favorable. Accordingly, it would be beneficial to mix vaccines in the same container, such as a flexible, disposable bag, that the vaccines will be shipped within.

Good mixing is critical for optimization of bioreactor processes. A well-designed mixing system provides three basic functions: creation of constant conditions (nutrients, pH, temperature, etc.) in a homogeneous distribution; dispersion of gas for supplying, e.g., oxygen, and extracting carbon dioxide where and when needed as in a bioreactor; and optimization of heat transfer. Also, providing acceptable mixing, without imparting damaging shear effects, becomes more challenging as the size and/or aspect ratio of the bioreactor container increases. Certain commercial mixer and bioreactor platforms include a single bottom mounted impeller. Single bottom impellers produce a vortex having stagnant zones, decreasing mixing. Multiple impellers and/or higher impeller speeds improve mixing. However, higher shear rates associated with multiple impellers and/or high impeller speeds, as well as some baffles, can damage cells within the container. A baffle may enhance mixing efficiency by disrupting the vortex.

Furthermore, it is often favorable to supply materials and/or processing aids, liquids or gases such as antifoam agents, nutrients, and/or oxygen to the system to promote cell growth within a container, bioreactor or bag. Typically, these materials are added either via a plurality of ports in the top and bottom of the container/bag, wherein the mixing element distributes them. However, this is an inefficient method for distribution in that the port is typically located along an inner surface of the container and distribution of the materials to where they are needed is often incomplete.

Last, various sensors are generally used in such bags to determine the state or condition of the liquid or cells within the bag. Such sensors typically monitor pH, dissolved gases, temperature, turbidity, conductivity, and the like to determine homogeneity of such properties throughout the bag. To do so, sensors are often placed within dip tubes from the top of the bag into the inner volume of the bag at one or more locations. Alternatively, sensors are simply mounted to the inner wall of the bag.

The dip tubes can create inconsistencies in the fluid flow in the bag, thereby complicating the mixing. Moreover, placement of the sensors in dip tubes in the bag before use presents difficulties as the dip tubes are rigid plastic sleeves that cannot be packed as efficiently, making transport and storage of the bag less than optimal. Furthermore, the use of sensors along the inner wall also limits the data that can collected and often what is occurring away from the inner wall of the bag must be inferred from the data obtained by the sensors, as opposed to direct measurements, which is an unfavorable method for data monitoring and collection.

It is an advance in the art to provide a disposable or single use container for fluids having an improved collapsible, baffle and/or baffle system that is transported easily, promotes homogeneous mixing, and provides a platform for more accurate sensor positioning. More efficient methods for delivering liquids and gases where efficacious, e.g., proximal to a desired position, e.g., a fluid surface level, within various depths of the fluid level, and/or near an impeller within the bag also represent advances in the art.

### SUMMARY

A collapsible container for a fluid that includes a flexible material, defining an internal working volume; at least one collapsible baffle adhered within the working volume of said collapsible container, the at least one baffle having one or more channels for delivering one or more fluids into the working volume via at least one hole in said one or more channels, one or more channels in said container for exiting or venting fluids from the working volume, and an impeller assembly disposed at least partially within said working volume of said container, substantially as shown in and/or described in connection with at least one of the figures, as set forth more completely in the claims. Various benefits, aspects, novel and inventive features of the present disclosure, as well as details of exemplary embodiments thereof, will be more fully understood from the following description and drawings.

Embodiments of the disclosure include a container for a fluid, comprising a volume formed of a flexible material, one or more inlets in said container, optionally, one or more outlets in said container, an impeller assembly mounted at least partially within said volume of said container, and a baffle in said volume of said container, said baffle being placed in said container so as to have a vertical component and/or both a horizontal and a vertical component in said container.

In accordance with some embodiments, disclosed herein is a container, bioreactor or bag, such as a disposable or single use container, optionally having one or more inlets and one or more outlets and a mixer associated with the container, to promote mixing, homogenizing, dispersion, and/or circulation of one or more ingredients contained or added to the container. In accordance with some embodiments, the container includes a collapsible baffle, for example, a flexible polymeric film baffle, placed within an interior volume of the container to improve processing. In accordance with some embodiments, the baffle is placed within the container to disrupt the vortex formed by the mixer and/or, in some embodiments, prevent formation of a vortex. Also, baffle(s) disclosed herein can be used deliver fluid into a more desirable flow pattern that includes both axial and/or radial flow. In accordance with some embodiments, the baffle is used with a single impeller to limit shear effects. In accordance with some embodiments, the baffle is shaped with vertical elements to enhance mixing against the inner wall of the bag. In accordance with some embodiments, the baffle is shaped with both horizontal and vertical elements to enhance disruption of the vortex across the vessel height and provide homogeneous mixing throughout operating volumes and any gradient or height associated therewith. In accordance with some embodiments, the baffle is X-shaped or ladder-shaped. In accordance with some embodiments, the baffle comprises, at least partially therethrough, a pathway, such as a channel or conduit, for the delivery of liquid or gas supplements, materials, and/or processing aids near or at a desired location of application and/or for the use of sensors at their desired location within an internal volume of the bag. In some embodiments according to the disclosure, tubing may be inserted within the channel or conduit. Also disclosed is a system for mixing a fluid in a container having an internal volume, the system comprising a container, an impeller assembly, a drive for the impeller assembly, and one or more baffles placed within the internal volume to enhance mixing and/or homogeneity of a fluid during mixing while providing a pathway within the baffle for the delivery of liquid or gas supplements near or at the desired location of application. According to some embodiments, sensors are placed at a desired location within the inner volume of the bag and/or within one or more baffle(s).

Improved methods for mixing a fluid or liquid in a container having an impeller assembly and a baffle placed in the container, the baffle comprising one or more channels/conduits for materials to be introduced into the container and/or for locating sensors within the container, are also disclosed. Some methods described herein comprise the delivery of a fluid into a container, wherein an impeller assembly is at least partially contained within the container and drives the blades or vanes of the impeller assembly to agitate the fluid in the container or bag. In some methods, the driver for the impeller assembly is external to the bag, and drives the impeller assembly magnetically. The baffle in the container improves mixing. In some methods, liquids and/or gases can be delivered through the channel(s)/conduit(s) of the baffle from the exterior of the container to a set point within the container and/or sensors can be located within the container at desired points, wherein measurements can be taken within at least one of a plurality of locations within the volume of the container.

Any and all embodiments of baffles and methods for processing using the baffles described herein are collapsible within the bag and may be expanded. When the baffles are at least partially expanded, the baffle(s) are capable of providing all functional features described herein. For example, this disclosure provides an integrated assembly that integrates channels/conduits for delivering various liquids and gases with the baffle(s) into a working volume of the container. The embodiments of the baffle(s) virtually eliminate piping or tubing typically required on a top of the bioreactor and/or the baffle(s) reduces the connection to the reactor to a lower portion of the reactor.

Also, any and all embodiments of baffles described herein facilitate the addition of various processing aids, e.g., antifoaming agents. For example, a conduit/channel of the baffle(s) is capable of delivering an anti-foaming agent onto the liquid surface of a, e.g., cell culture through a hole or a plurality of holes in the baffle(s). The hole(s) enable optimum placement and distribution of the antifoam agent, typically a liquid, in multiple places throughout the foam layer residing on the surface of the liquid being processed, wherein the effectiveness of the antifoam addition is increased. The hole(s) also permit better efficiency of antifoam action by allowing broader distribution of antifoam liquid at multiple locations on the liquid surface where foam accumulates. Also, as antifoam agents are distributed at multiple locations through a single baffle, i.e., less external tubing is required, saving manufacturing costs and/or set up times, simplifying the plumbing, i.e., the number of hoses, connectors, placement of hoses and connectors, etc., are reduced and easier to set up and operate. Any and all embodiments of baffles described herein reduce the number of separate tubes and connectors for delivering gas(es), feeds, and/or processing agents. Hoses and/or tubing for liquid/gas addition and/or venting typically run externally from the top of the bioreactor/bag/container, often extending to the user at ground level. Some bags may also include a form of internal hard piping to direct flow. These are typically single pipes or tubing lines tied together with tie wraps or some other means to make them manageable to the user. In a typical bioreactor application, many connections to the system are connected to the top of the bioreactor. Embodiments of the baffle(s) described herein comprise connections that are made to the lower part of the bag. Furthermore, the hose/tube connections described herein run into the top of the bag and are integral to the baffle and, therefore, eliminate the need for most external hoses/tubing. The integrated assembly of the bag and tubing described herein provide all the functionality of managing and organizing the tubing as well as bringing the interaction with the user to a lower or ground level, without external tubing reaching down from the top of the bag, combined with a baffle function.

Furthermore, the channels/conduits within the baffle enable a liquid feed to be delivered through the bag wall, up and/or through the baffle(s), and into the top of the reactor/bag. Also, the liquid feed can traverse the baffle(s) such that the feed is introduced to the cell culture in a high flow area (for example, near an impeller(s)). Delivering the liquid feed(s) below the liquid surface of the cell culture enhances mixing efficiencies. Delivery, for e.g., of an anti-foaming agent above the liquid surface enhances anti-foam agent efficiency. The baffle(s) having hole(s) permit faster mixing upon delivery because the feed is not necessarily dripped in at the liquid surface in a single stream.

Similarly, a first conduit or, optionally, a second conduit within the baffle(s) enables gas(es) to be introduced through the bag wall, and up the baffle to a head space at the top of the bag or bioreactor, i.e., above a fluid or liquid level within the bag. Gases can then exit via a port, which optionally comprises a vent filtration device. Incorporating these egress paths from the headspace through the bioreactor/bag container walls permits user interaction with the vent at a ground level (as opposed to requiring a ladder or step stool to reach the headspace area where vents are typically located). Also, sparge gases may be introduced into the wall of the bag and channeled to the baffle(s). Gas(es) may be introduced through a hole or plurality of holes in the baffle(s) to create an open pipe sparger or, alternatively, a ring-type sparger. The flow path is routed above the liquid surface, wherein an air break is created to prevent liquid from draining when the gases are not present, obviating the need for a check valve. The baffle having holes enables the manufacture of a sparger by putting a series of holes along one or both sides of the lowest horizontal baffle. This sparger can be manufactured for little to no extra cost. Gases may be delivered through filters as they enter the flowpath to assure sterility.

The shape of and/or the mounting points of the collapsible baffle enable it to collapse for shipping and storage while providing rigidity while the bioreactor or bag or mixer while in use. Optionally, rigid members may be placed within the baffle(s) to enhance rigidity.

### BRIEF DESCRIPTION OF THE DRAWINGS

FIG. 1 is an upper perspective view of a container having a baffle, according to some embodiments described in the disclosure;

FIG. 2 is a cross-sectional view, taken along line 2-2, of three embodiments of the baffle shown in FIG. 1, according to some embodiments described in the disclosure;

FIG. 3A is a X-shaped baffle disposed within a container, according to some embodiments described in the disclosure;

FIG. 3B depicts a close up-view of a portion of the X-shaped baffle of FIG. 3A, according to some embodiments described within the disclosure;

FIG. 4 comprises a first multi-member baffle, according to some embodiments described in the disclosure; and

FIG. 5 comprises a second multi-member baffle, according to some embodiments described in the disclosure.

### DETAILED DESCRIPTION

So the manner in which the features disclosed herein can be understood in detail, a more particular description of the embodiments of the disclosure, briefly summarized above, may he had by reference to the appended drawings. It is to be noted, however, that the appended drawings illustrate only typical embodiments of this invention and are therefore not to be considered limiting of its scope, for the invention may admit to other equally effective embodiments. It is also to be understood that elements and features of one embodiment may be found in other embodiments without further recitation and that, where possible, identical reference numerals have been used to indicate comparable elements that are common to the figures.

It is to be understood that recitation to a bag, container, and bioreactor refers to any flexible container capable of processing biological fluids, growing cells, fermenting, and the like and are used interchangeably throughout except where context dictates otherwise. It is also to be understood that the term horizontal indicates a feature that is substantially parallel with a plane of the horizon. The term vertical connotes a feature having an axis substantially at right angles to the horizontal. A feature described as having both vertical and horizontal features can indicate, for e.g., a +-shape and/or a diagonal shape, for e.g., an X-shape.

In accordance with certain embodiments, the disposable container is designed to receive and maintain a liquid or a fluid. In some embodiments, the disposable container comprises monolayer walls or multilayer flexible walls formed of a polymeric composition such as polyethylene, including ultrahigh molecular weight polyethylene, very low density polyethylene, ultralow density polyethylene, linear low density polyethylene, low density or medium density polyethylene; polypropylene; ethylene vinyl alcohol (EVOH); polyvinyl chloride (PVC); polyvinyl acetate (PVA); ethylene vinyl acetate copolymers (EVA copolymers); thermoplastic elastomers (TPE), and/or blends or alloys of any of the foregoing materials as well as other various thermoplastics materials and additives known to those in the art. The disposable container, owing to the materials from which it is manufactured, is collapsible and expandable. The disposable container may be formed by various processes including, but not limited to, co-extrusion of similar or different thermoplastics; multilayered laminates of different thermoplastics; welding and/or heat treatments, heat staking, calendaring, or the like. Any of the foregoing processes may further comprise layers of woven or non-woven substrates, adhesives, tie layers, primers, surface treatments, and/or the like to promote adhesion between adjacent layers. By “different,” it is meant different polymer types such as polyethylene layers with one or more layers of EVOH as well as the same polymer type but of different characteristics such as molecular weight, linear or branched polymer, fillers and the like, are contemplated herein. Typically, medical grade polymers and, in some embodiments, animal-free plastics are used to manufacture the containers. Medical grade polymers may be sterilized, for e.g., by steam, ethylene oxide or radiation, including beta and/or gamma radiation. Also, most medical grade polymers are specified for good tensile strength and low gas transfer. In some embodiments, the polymeric material is clear or translucent, allowing visual monitoring of the contents and, typically, are weldable and unsupported. In some embodiments, the container may be a bioreactor capable of supporting a biologically active environment, such as one capable of growing cells in the context of cell cultures. In some embodiments, the container may be a two-dimensional or “pillow” bag or, alternatively, the container may be a three-dimensional bag. The particular geometry of the container is not limited in any embodiment disclosed herein. In some embodiments, the container may include a rigid base, which can provide access points such as ports or vents. Any container described herein may comprise one or more inlets, one or more outlets and, optionally, other features such as sterile gas vents, spargers, and ports for the sensing of the liquid within the container for parameters such as conductivity, turbidity, pH, temperature, dissolved gases, e.g., oxygen and carbon dioxide, and the like as known to those in the art. The container is of a sufficient size to contain fluid, such as cells and a culture medium, to be mixed from bench-top scale to 3000L bioreactors.

In some embodiments, the container may be a disposable, deformable, foldable bag that defines a closed volume, is sterilizable for single use, capable of accommodating contents, such as biopharmaceutical fluids, in a fluid state, and can accommodate a mixing device partially or completely within the closed volume of the container, e.g., an internal working volume. In some embodiments, the closed volume can be opened, such as by suitable valving, to introduce a fluid into the volume, and to expel fluid therefrom, such as after mixing is complete.

In accordance with some embodiments, the container comprises at least one collapsible baffle, the baffle being placed in the container such that when the container contains fluid and an impeller assembly is operating, enhanced (as opposed to when embodiments of the baffles described herein are not present) mixing of a fluid occurs. During mixing, a vortex is formed. Without intending to be bound by theory, it is believed that a vortex prevents, or at least slows, adequate mixing. It is believed that the disruption of the vortex, caused by the baffle, promotes more efficient and faster mixing. It is further believed that an appropriately designed baffle is capable of disrupting vortices without introducing unfavorable levels of shear, which would otherwise damage cells and/or biological fluids being processed. The inventors have surprisingly discovered that baffles are suitable for delivering processing aids, such as anti-foaming agents, at different fluid surface levels (where foam typically forms) while disrupting vortices. The inventors have also surprisingly discovered that baffles are suitable for delivering gases to various depths within a volume of fluid, obviating the need for a separate sparger. The baffles are further capable of delivering feed additions for the cell culture in a fluid at various depths within a container. For example, delivering feed additions proximal to a high flow area, e.g., near an impeller. The baffles may also house supporting members to stiffen the baffles during processing.

In some embodiments, the baffle has at least a vertical component submerged in the fluid of the container. In some embodiments, the baffle comprises both horizontal and vertical components submerged in the fluid of the container. In accordance with some embodiments, the baffle has a vertical component and is attached at one or more portions of an inner sidewall of the container. In accordance with some embodiments, the baffle has one or more horizontal components and is attached at one or more portions of an inner sidewall of the container. In accordance with some embodiments, the baffle has a vertical component and a horizontal component so that one end of the baffle is attached either to a bottom surface or the sidewall of the container and an opposite end of the baffle is attached to the top surface or a different portion of the sidewall than the first end. In accordance with some embodiments, a portion less than the entire baffle is submerged in the fluid during use/processing of a biological fluid. In accordance with some embodiments, the baffle extends to the inner radial dimensions of the container. In accordance with some embodiments, the baffle is X-shaped. In accordance with some embodiments, the baffle comprises a ladder-shape, e.g., two or more vertical members connected with one or more horizontal members. In accordance with some embodiments, the baffle(s) may be oriented upside up or upside down.

In accordance with some embodiments, the baffle provides one or more channels/conduits through an interior volume of the baffle for the delivery of materials into or out of the container. In accordance with some embodiments, the baffle provides one or more channels/conduits through an interior volume of the baffle for the location of sensors within a desired position within the container. In some embodiments, the sensors are single use sensors. In accordance with some embodiments, the baffle is in the form of a flexible plastic sleeve having an outer sealed surface and an inner volume. In accordance with some embodiments, the baffle is formed of a single piece of plastic folded in two portions to form the outer surface and inner volume. In accordance with some embodiments, the baffle is formed of two or more pieces of plastic to form the outer surface and inner volume. In accordance with some embodiments, the inner volume itself forms the channels/conduits. In some embodiments, the inner surfaces of the inner volume are sealed to each other to form distinct pathways. In accordance with some embodiments, the baffle is formed by a diecutting operation that stamps two similarly shaped portions of film, heat staking them to form the finished baffle. In further embodiments, pieces of tubing form the channels/conduits. In further embodiments, pieces of tubing are disposed within the channels/conduits.

In some embodiments, each container contains, either partially or completely within its internal working volume, an impeller assembly for mixing, dispersing, homogenizing, and/or circulating one or more liquids, gases and/or solids contained in the container. The impeller assembly may include one or more blades, which are movable, such as by rotation or oscillation about an axis. The impeller assembly converts rotational motion into a force that mixes the fluids in contact therewith. The impeller assembly may be formed in the top of the container and via a shaft extend downward into the container volume. The shaft is connected to a motor outside of the container and the shaft has one or more impeller blades on it. Such assemblies are often referred to as “lightning-style” assemblies. Also, in some embodiments, the impeller assembly can be formed in a bottom portion of the container and is connected to a motor by a direct shaft to a motor outside the container or, alternatively, is magnetically coupled to the motor so no shaft needs to penetrate through the container wall.

Proper design and implementation of the impeller/baffle combination provides a mixing solution across a wide range of volumes and aspect ratios with the ability to provide better delivery of materials to the container and/or better location of sensors within the volume of the container, enabling the development of a family of bioreactor or mixer systems with excellent scalability and well-defined performance. Furthermore, each of the containers and baffles contemplated herein are made of thin, compliant plastics materials and, therefore, are collapsible for easy packing, unpacking, transit, and disposal. In some embodiments, the bioreactor, bag, and/or container comprises a collapsible dip tube. The collapsible dip tube may be a conduit projecting from the bag for removing fluid from the bag. The collapsible dip tube may be made of a flexible, compliant material. For example, the collapsible dip tube may be manufactured from any of the polymers or materials discussed herein. Furthermore, the collapsible dip tube may be removably attached to the bag. In some embodiments, the collapsible dip tube is an integral part of the bag. In this context, integral indicates that the collapsible dip tube could not be removed from the bag without destroying either the bag or the collapsible dip tube. Furthermore, the collapsible dip tube may be used in, for example, perfusion processes. Perfusion is a process for maintaining a cell culture within a bioreactor. Perfusion processing comprises steps in which substantially equivalent volumes of media are added and removed from the bioreactor while the cells are retained in the reactor, either in suspension or on microcarriers. A steady source of fresh nutrients and constant removal of cell waste products is attained using perfusion.

FIG. 1 is an upper perspective view of a container 10 having a baffle 18, according to some embodiments described in the disclosure. The container 10 has an impeller assembly 28, further comprising a base 14 and one or more moveable blades or vanes 16, wherein the container 19 is disposed within a shell 5. The container 10 may be a two-dimensional or “pillow” bag style container or, alternatively, a three-dimensional bag. In some embodiments, the container 10 has a minimum internal working volume of 200 L, and a maximum internal working volume of 3000 L. It is to be understood that, irrespective of size, the container 10 need not be at full liquid capacity to operate. For example, any container 10, whether 200 L or 3000 L may operate at a maximum internal working volume H or, alternatively, a minimum internal working volume L, which is at a liquid height just above the impeller assembly 28. The container 10 may also operate at any working internal volume between the maximum working volume H and the minimum working volume L. In some embodiments, at least a portion of the impeller assembly 28 is disposed within the internal working volume 32 of the container 10. In some embodiments, the driver, such as a motor (not shown) for the impeller assembly 28, is external to the container 10.

The container 10 may have a relatively flat bottom B or, alternatively, a conical bottom (not shown) or other tapered bottom. The container 10 may, alternatively, comprise a two-dimensional tapered bottom (not shown). The number and shape of the blades 16 of the impeller assembly 28 is not particularly limited, provided the blades 16 are capable of sufficiently agitating a fluid within the container 10 when actuated. The blades may be constructed of plastic material, such as polyethylene, or any polymer resistant to gamma irradiation, such as polypropylene or a polypropylene co-polymer, for sterilization purposes. The shell 5 optionally comprises a base 14, which may be constructed of plastic material, such as polyethylene, or any polymer resistant to gamma irradiation, such as polypropylene, or a polypropylene co-polymer, also for sterilization purposes.

In some embodiments, the base 14 includes an axially extending member 22. The axially extending member 22 accommodates the magnetic base of the impeller assembly 28, such as a mixing impeller overmolded magnet (not shown), wherein the blades 16 extend axially above the member 22 and are free to rotate when the magnetic impeller is driven by a drive magnet. In some embodiments, wherein the impeller assembly 28 is installed in the container 10, the extending member 22 protrudes outside the container 10, wherein the base 14 is sealed to the container 10. The remainder of the impeller assembly 28 is housed inside the container 10. In some embodiments, the impeller assembly 28 is placed at or near the bottom B of the container 10, wherein the container 10 is in mixing position (such as a hanging position) and proximal to at least one port 46, such outlet 30 of the container 10.

As shown in FIG. 1, the container 10 is made of a compliant, weldable plastic such as polyethylene or other polymers or layered constructions of polymer(s) described herein, and is sealed, forming a closed vessel having an internal working volume 32. Fluid access into the internal working volume 32 is via one or more of a plurality of ports 46. The plurality of ports 46 are, optionally, adhered, sealed, or otherwise welded directly to the container 10. Each or any of the plurality of ports 46 may comprise a plug (not shown) or have a conduit or tube 44 attached or formed integrally therewith. In some exemplary embodiments, the tube(s) 44 are formed of a silicone material, which is suitable of sterilization via radiation. One or more ports 46 may also provide access to the inner volume 26 of the baffle 18. Liquid or gas can be delivered into the baffle 18 via ports 46 and subsequently into the container 10. For example, the baffle 18 comprises one or more hole(s) or orifice(s) 38 for allowing a liquid or a gas to pass therethrough into the working volume 32. In some embodiments, the hole(s) 38 have a diameter ranging from 0.1 mm to 3.0 mm. In some exemplary embodiments, the hole(s) 38 comprise a diameter of 0.5 mm to 1.0 mm. In some exemplary embodiments, the hole(s) 38 comprise diameters forming a gradient, e.g., the holes get progressively larger or smaller from a proximal end to a distal end along the baffle(s). In some embodiments, the ports 30, 40, 50 may comprise a one-way valve (not shown) or a hydrophobic membrane (not shown) so that liquid (optionally, with the valve) or gas (optionally, with the valve or hydrophobic membrane) can only selectively enter or exit through the port 30, 40, 50 as may be desired. It is further noted that fluid can exit the container via ports 30. For example, the container 10 comprises a plurality of exit ports 30 proximal the Bottom B of the container 10. The container 10 further comprises a plurality of upper inlet ports 50 proximal the top of the container 10. The container 10 further comprises a plurality of baffle inlets 40 in fluid communication with the baffle(s) 18.

In some embodiments, the exit ports 30, the upper inlet ports 50 and/or the plurality of inlet baffle inlets 40 comprise a one-way valve (not shown) or a hydrophobic membrane (not shown) so that liquid (with the valve) or gas (with the valve or hydrophobic membrane) can only selectively enter or exit therethrough, as may be desired.

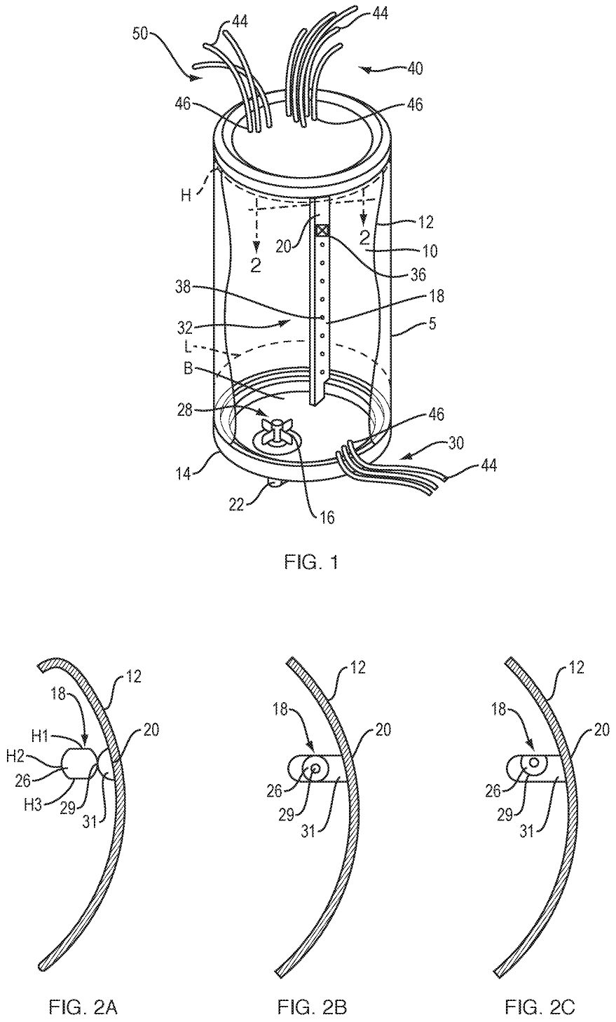

One or more baffle(s) 18 are formed along the inner surfaces of the sidewall(s) 12 of the container 10. The baffle(s) 18 are in the form of a flexible plastic sleeve (shown below) having an outer sealed surface (shown below) and an inner volume isolated from the remainder of the container 10 as shown in FIG. 2. The one or more baffle(s) 18 may comprise a valve 36. In some embodiments, the valve 36 may be a ball valve or a needle valve in communication with a controller. In some exemplary embodiments, the valve 36 is a check valve. A check valve may be specified to prevent back flow of a fluid when pressure is low, for example, approximately twenty (20) psi or 140 kPa or less. In some exemplary embodiments, the check valve is specified for five (5) psi or 35 kPa or less.

FIG. 2 is a cross-sectional view, taken along line 2-2, of three embodiments of the baffle 18 shown in FIG. 1, according to some embodiments described in the disclosure. As shown in FIGS. 2A-2C, the baffle(s) 18 are hollow structure(s) and comprises an inner volume that forms the pathway 26. In some embodiments, the sleeve 20 of the baffle(s) 18 is bonded or sealed into a seam of the sidewall 12 where it is located. The sleeve 20 may also be sealed such as by heat bonding or adhesive as a separate piece to the sidewall 12 as shown in FIG. 1. It is to be understood that at least three embodiments of baffle 18 are contemplated herein. FIG. 2A comprises a first embodiment of the baffle 18, according to some embodiments. The first embodiment of the baffle 18, as depicted, comprises a baffle pocket 31. The baffle pocket 31 is capable of receiving means for becoming rigid. For example, means for becoming rigid comprises removably placing a rigid rod (not shown) within the baffle pocket 31. The baffle pocket(s) 31 extend through at least a portion of the length of the baffle 18. In some embodiments, the pocket(s) 31 are tapered from a narrowest point adjacent the bottom wall to a widest point adjacent the top wall. The rigid rod may comprise a plastic material, steel, wood, or any suitable rigid material. In some embodiments, the means for becoming rigid is selected from the group consisting of a rigid insert that is placed within the baffle pocket(s) formed within at least a portion of the pocket(s) 31 and a baffle(s) 18 formed across a portion of the internal working volume of the bag 10, the baffle(s) 18 having a first portion attached to a first portion of the sidewall 12 and the baffle(s) 18 having a second portion attached to a second portion of the sidewall 12 at least tangentially across from the first sidewall portion, wherein the baffle(s) 18 is capable of being drawn tight upon filling of the bag 10. The first embodiment of baffle 18 also comprises a conduit 26. In some embodiments, the one or more baffle pockets 31 extend from a first position adjacent the bottom wall on the one or more sidewalls 12 to a second position on one more of the sidewalls 12 adjacent a top wall at least tangentially opposite that of the first position.

The conduit 26 is capable of delivering a gas or fluid into the working volume of the container or bioreactor, as described above, via a hole or a plurality of holes 38. The hole(s) 38 may be placed along a longitudinal axis of the baffle 18. Furthermore, the hole(s) 38 may be placed along any combination of locations. For example, the hole(s) 38 may be disposed along a first axial surface H1, a second axial surface H2, and/or a third axial surface H3. A seal 29 is disposed between the conduit 26 and the baffle pocket 31. The seal 29 may comprise, for e.g., a heat seal or heat staking or other method of crimping the baffle 18 into two liquid tight portions. FIG. 2B comprises a second embodiment of the baffle 18, according to some embodiments. The second embodiment of the baffle 18 comprises a baffle pocket 31. As described above, the baffle pocket 31 is capable of receiving means for becoming rigid. For example, means for becoming rigid comprises removably placing a rigid rod (not shown) within the baffle pocket 31. The rigid rod may comprise a plastic material, steel, wood, or any suitable rigid material. The first embodiment of baffle 18 also comprises a conduit 26. The conduit 26 is capable of delivering a gas or fluid into the internal working volume of the container or bioreactor, as described above, via a hole or a plurality of holes (as described above). A seal 29 is disposed between the conduit 26 and the baffle pocket 31. The seal 29 may comprise, for e.g., a heat seal or heat staking or other method of crimping the baffle 18 into two portions. FIG. 2C comprises a third embodiment of the baffle 18, according to some embodiments. The baffle(s) 18 are formed by diecutting two similar pieces of polymer and heat-staking the two pieces along various surfaces, melting the polymer and creating a fused area.

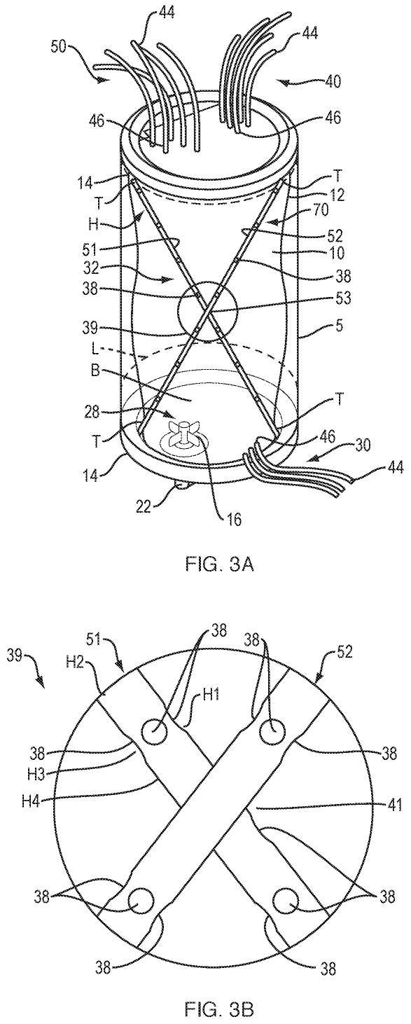

FIG. 3A is a X-shaped baffle 70 disposed within a container 10, according to some embodiments described in the disclosure. FIG. 3A depicts a container substantially similar to FIG. 1. As shown, the baffle 70 comprises two portions that extend from a portion of the sidewall 12 and extend inwardly into the volume 32 of the container 10 and sealed off to form the baffle 70. In some embodiments, the baffle 70 is a film made of weldable plastic, e.g., polyethylene, low density polyethylene, high density polyethylene, linear low density polyethylene, and other suitable grades of polyethylene as are known to those in the art. The baffle 70 includes a first leg 51 and a second leg 52. The first leg 51 and the second leg 52 intersect and, optionally, attaches to the first leg 51. In some embodiments, the location of the attachment of the first leg 51 and the second leg 52 is at, approximately, the longitudinal midpoint 53 of the first leg 51 and the second leg 52. However, it is not necessary that the first leg 51 and the second leg 52 actually contact or attach to one another. Each terminal end T of each of the first leg 51 and the second leg 52 is bent at approximately a 30° to 60° angle with respect to the main body of each leg. In some exemplary embodiments, each terminal end T of the first leg 51 and the second leg 52 is bent at a 45° angle with respect to the main body of each leg.

In some embodiments, each of these terminal ends T can be affixed to the internal wall 12 of the container 10, such as by welding or heat staking, to affix the baffle 70 in place in the container 10, wherein the first leg 51 and the second leg 52 are attached to the bag without being affixed to one another. In some embodiments, where the container 10 is a bag, the terminal ends T are heat sealed or welded within the seams of the bag.

The baffle 70 comprises hole(s) 38. In some embodiments, the baffle 70 is approximately 12.0 cm to 75.0 cm in width and is approximately 0.125 mm to 0.400 mm in thickness. In some embodiments, the hole(s) 38 have a diameter ranging from 0.10 mm to 3.0 mm. In some exemplary embodiments, the hole(s) 38 comprise a diameter of 0.50 mm to 1.0 mm. In some exemplary embodiments, the hole(s) 38 comprise diameters forming a gradient, e.g., the holes get progressively larger or smaller from a proximal end to a distal end along the baffle(s) 70. The hole(s) 38 may be placed along an axial length of the baffle 70. Furthermore, the hole(s) 38 may be placed along any combination of locations. For example, the hole(s) 38 may be disposed along a first axial surface H1, a second axial surface H2, a third axial surface H3, and/or a fourth axial surface H4, that is opposite the second axial surface H2. As is evident, the hole(s) 38 need not be coaxial along both a longitudinal axis of the baffle 100 and a lateral axis of the baffle 70.

FIG. 3B depicts a close up-view 39 of portion of the baffle 70 of FIG. 3A, according to some embodiments described within the disclosure. As above, the hole(s) 38 may be placed along an axial length of the baffle 70. As described above, the hole(s) 38 may be placed along any combination of locations. For example, the hole(s) 38 may be disposed along a first axial surface H1, a second axial surface H2, a third axial surface H3, and/or a fourth axial surface H4 that is opposite the second axial surface H2. Also, as described above, the upper legs 51, 52 may be adhered to one another at junction 41, for example, by heat staking, welding, etc., or, alternatively, the upper legs 51, 52 may be separate parts of two different baffles.

As exemplified in FIG. 1, in some embodiments, these seams line up behind the impeller 28 (12 o'clock) and across the bag at 6 o'clock. The bottom is attached at the lowest level of the bag and the top at a level that is above the maximum internal working volume 32 of the bag. Other attachment locations are possible, including attaching the baffle 70 directly to the base of the system that supports the container 10, and/or to the top of the container 10 instead of the sides. In some embodiments, “slack” is introduced in the baffle(s) 18, 70, which may be acceptable. By way of explanation and not limitation, the legs of the baffle(s) 18, 70, attached to the bag, need not be taut. Irrespective of attachment locations, in some embodiments, the upper legs 51, 52 extend out of a fluid being processed, i.e., above the maximum internal working volume 32 of the bag (as opposed to being fully immersed in the fluid). As above with respect to baffle 18, the baffle X-shaped 70 comprises orifice(s) or hole(s) 38 for delivery of gases, fluids and/or processing aids to traverse into the working volume 32. For example, the container 10 comprises a plurality of exit ports 30 proximal the Bottom B of the container 10. The container 10 further comprises a plurality of upper inlet ports 50 proximal the top of the container 10, which may be in fluid communication with the baffle 100 via upper leg 51. The container 10 further comprises a plurality of baffle inlets 40 in fluid communication with the baffle(s) 70 via upper leg 52. Mixing time is reduced by, at least, approximately 50%, according to embodiments of the baffle(s) 18, 70 in conjunction with various containers having varied aspect ratios.

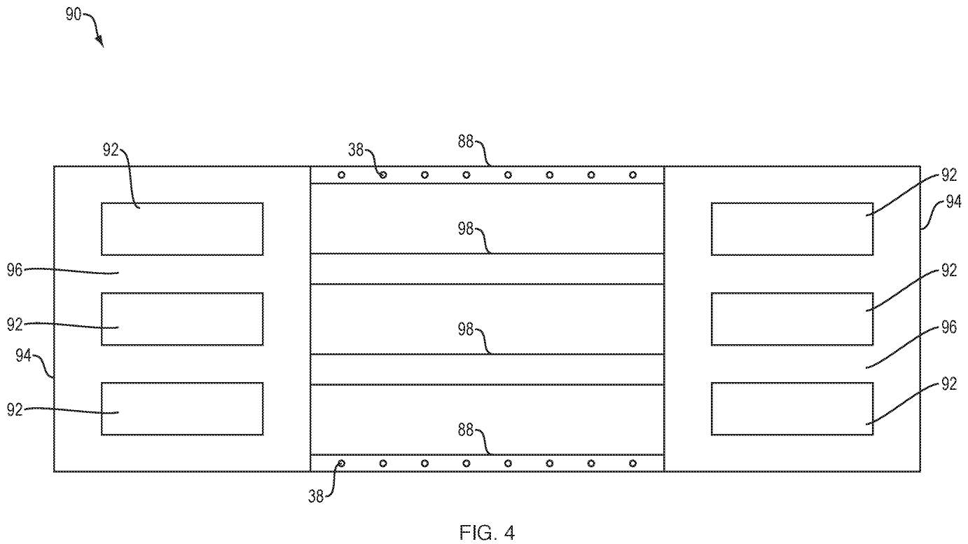

FIG. 4 is a multi-member baffle 90, according to some embodiments described in the disclosure. In FIG. 4, the baffle 90 is a ladder-shaped baffle and comprises more than one channel for delivering gases, liquids, feeds, etc., within the same baffle 90. In some embodiments, the channels are formed by heat sealing or adhering portions of the baffle 90 together, forming separate and distinct pathways to different locations or to carry different materials or sensors (not shown) of the container 10. Also, having one baffle 90 that comprises more than one channel reduces the amount of tubes and connectors.

The baffle 90 is collapsible within a bag, container or bioreactor (not shown). The baffle 90 is made of a polymer and adheres to the sidewalls of the bag, container, or bioreactor (also collapsible) as described above with respect to the bag 10. The baffle 90 comprises side rails 94. The side rails 94 may attach, be heat staked, welded, etc., to the bag, container, or bioreactor. The baffle 90 further comprises windows 92 and intermediate portions 96, which can disrupt a vortex during processing, enhancing mixing. The baffle 90 also comprises at least one fluid delivery member 88 having hole(s) 38 and at least one non-fluid delivery member 98. In this context, a fluid is understood to be a gas(es), a liquid(s), and/or a liquid feed for cells. The at least one fluid delivery member 88 is in fluid communication via a port (not shown) for delivery of a fluid. As described above, the hole(s) 38 permit entry of the fluid into the working volume of the bag, container, or bioreactor. For example, the at least one fluid delivery member 88, shown above the non-fluid delivery member 98, may be used to deliver an anti-foam agent to the working volume of the bag. In some exemplary embodiments, the at least one fluid delivery member 88 would be oriented above a liquid level within the bag. In some embodiments, the at least one fluid delivery member 88, shown below the non-fluid delivery member 98, may be used to deliver, for example, gases such as oxygen and/or carbon dioxide to the working volume of the bag. It is to be understood that any of the fluid delivery members 88 and/or the non-fluid delivery members 98 are collapsible and capable of disrupting a vortex within the bag. It is to be understood that any of the fluid delivery members 88 and/or the non-fluid delivery members 98 of the baffle 90 nay further comprise any of the embodiments as formed in FIGS. 2A-2C to provide two or more channels, at least one of which can have a rigid member placed therein for providing support to the baffle 90 during operation.

The baffle 90 may further comprise a port of exiting fluids, typically proximal to the bottom of the bag. The baffle 90 may further comprise a port for exiting gases to vents, typically proximal to the top of the bag. In some embodiments, the hole(s) 38 within the baffle 90 have a diameter ranging from 0.10 mm to 3.0 mm. In some exemplary embodiments, the hole(s) 38 comprise a diameter of 0.5 mm to 1.0 mm. In some exemplary embodiments, the hole(s) 38 comprise diameters forming a gradient, e.g., the holes get progressively larger or smaller from a proximal end to a distal end along a longitudinal axis of the baffle(s) 90.

In some embodiments, the baffle(s) 18, 70, 90 is placed in the container such that it extends through the vortex (or the region where the vortex would form in the absence of the baffle(s) 18, 70, 90) at a given fluid level. The position of the vortex changes with aspect ratio of the container 10. The region where the vortex would form in the absence of the baffle(s) 18, 70, 90 can be determined from experience, or by mixing fluid in the container under similar mixing conditions that will be used in operation, but in the absence of the baffle(s) 18, 70, 90 and noting where the vortex forms. A “vortex map” can be created, documenting the location of the vortex for a given container aspect ratio, container volume, impeller position and impeller size. For an aspect ratio of 1:1 in a 1000 L container, the vortex is generally located at the 6 o'clock position. For an aspect ratio of 2:1 in a 2000 L container, and for an aspect ratio of 1.6:1 in a 200 L container, the vortex is generally located at the 9 o'clock position. Any embodiment of any baffle(s) 18, 70, 90 may comprise an inner volume, such as inner volume 26, described above, for delivering a fluid.

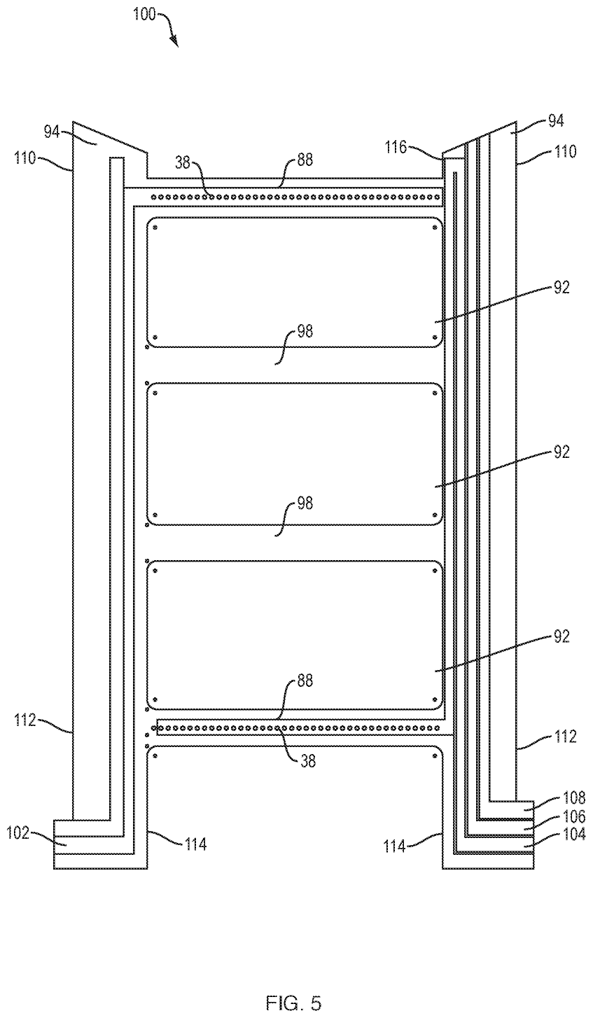

FIG. 5 comprises a second multi-member baffle 100, according to some embodiments described in the disclosure. The baffle 100 is a ladder-shaped baffle and made of one or more polymeric materials and adheres to the sidewalls of the bag, container, or bioreactor (also collapsible) as described above with respect to the bag 10. The baffle 100 comprises side rails 94. The side rails 94 may attach, be heat staked, welded, etc., to the bag, container, or bioreactor. The baffle 100 may also be staked or welded to the bag at, for example, an upper point 110 and a lower point 112 or any point therebetween.

The baffle 100 further comprises windows 92. The baffle 100 also comprises at least one fluid delivery member 88 having hole(s) 38 and at least one non-fluid delivery member 98. In some embodiments, the baffle 100 comprises an upper fluid delivery member 88, a lower fluid delivery member 88, and a plurality of non-fluid delivery members 98 disposed therebetween. As above, a fluid is understood to be a gas(es), a liquid(s), and/or a liquid feed for cells. The at least one fluid delivery member 88 is in fluid communication via a port (not shown) for delivery of a fluid. The baffle 100 comprises an upper fluid delivery member 88 that includes a channel 102 for delivery of the fluid to hole(s) 38, which would typically be above a liquid surface within the bag. The baffle 100 comprises a lower fluid delivery member 88 that includes a channel 104 for delivery of a fluid to hole(s) 38. The channel 104 traverses a lower portion 114 of the baffle 100, extends to an upper portion 116, which, as shown, is above the fluid delivery member 88, and terminates at the lower fluid delivery member 88. The lower fluid delivery member 88 is capable of delivering any fluid, gas or liquid, into the working volume of the bag. Also, the channel 104 need not extend above the upper fluid delivery member 88. So long as the channel 104 extends at least as high as the fluid level in the bag (which can be lower than upper fluid delivery member 88), a gas can be delivered to the working volume without having a check valve (or any other valve) disposed therein, i.e., no fluid can back up and out the channel 104. The baffle 100 further comprises additional channels. For example, channels 106, 108 are contemplated herein. Channels 106, 108 traverse from the lower portion 114 to the upper portion 116 of the baffle 100. Either of channels 106, 108 can be used to deliver gases to a space above the surface of a liquid within the working volume of the bag. Also, either of channels 106, 108 can be used to vent gases from above the liquid surface. Because channels 102, 104 terminate above a liquid surface within the bag, no check valve is needed. Furthermore, because all supply ports (not shown) to be used in conjunction with channels 102, 104, 106, 108 are all at or proximal to ground level, set ups, breakdowns, etc., are easier for an operator, i.e., step ladders for larger bags are not required. It is contemplated herein that the baffle 100 has all ports at a lower portion 114. It is within the scope of embodiments of the disclosure that more than two fluid delivery members 88 may be incorporated within the baffle 100. Similarly, it is within the scope of embodiments of the disclosure that more than two non-fluid delivery members 98 may be incorporated within the baffle 100. Furthermore, it is within the scope of embodiments of the disclosure that the fluid delivery members 88 and the non-fluid delivery members 98 may comprise a staggered orientation. In other words, the baffle 100 may have a fluid delivery member(s) 88 followed by a non-fluid delivery member(s) 98, followed by a fluid delivery member(s) 88 and subsequently another non-fluid delivery member(s) 98. Also, some embodiments according to the invention contemplate that the baffle 100 can be oriented and welded within a bag upside down, i.e., all ports disposed proximal a top of a bag. Also, as in FIG. 4, t is to be understood that any of the fluid delivery members 88 and/or the non-fluid delivery members 98 of the baffle 100 nay further comprise any of the embodiments as formed in FIGS. 2A-2C to provide two or more channels, at least one of which can have a rigid member placed therein for providing support to the baffle 100 during operation.

As described above, the hole(s) 38 permit entry of the fluid into the working volume of the bag, container, or bioreactor. For example, the at least one fluid delivery member 88, shown above the non-fluid delivery member 98, may be used to deliver an anti-foam agent to the working volume of the bag. In some exemplary embodiments, the at least one fluid delivery member 88 would be oriented above a liquid level within the bag. In some embodiments, the at least one fluid delivery member 88, shown below the non-fluid delivery member 98, may be used to deliver, for example, gases such as oxygen and/or carbon dioxide to the working volume of the bag, replacing and obviating the need for a separate sparger. It is to be understood that any of the fluid delivery members 88 and/or the non-fluid delivery members 98 are collapsible and capable of disrupting a vortex within the bag. It is to be understood that any of the channels 102, 104, 106, 108, and additional channels, as appropriate, are capable of housing a rigid member, as discussed above, e.g., pockets, for supporting the baffle 100 while in use. It is to be further understood that the pockets may be tapered from a narrowest point adjacent the bottom wall of the container to a widest point adjacent the top wall of the container.

The baffle 100 may further comprise a port for exiting fluids, typically proximal to the bottom of the bag. The baffle 100 may further comprise a port for exiting gases to vents, typically proximal to the top of the bag. In some embodiments, the hole(s) 38 within the baffle 100 have a diameter ranging from 0.10 mm to 3.0 mm. In some exemplary embodiments, the hole(s) 38 comprise a diameter of 0.5 mm to 1.0 mm. In some exemplary embodiments, the hole(s) 38 comprise diameters forming a gradient, e.g., the holes get progressively larger or smaller from a proximal end to a distal end along a longitudinal axis of the baffle(s) 100.

The one or more of the fluid delivery members 88 and/or non-fluid delivery members 98 of the baffle 100 may be disposed just above the liquid level where the foam forms. Smaller holes 38 disposed along the baffle can provide enhanced action as a light drip from a plurality of holes permits the use of lesser amounts of anti-foam agents.

All ranges for formulations recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described in connection with the embodiment is included in at least one embodiment of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Although some embodiments have been discussed above, other implementations and applications are also within the scope of the following claims. Although the specification describes, with reference to particular embodiments, it is to be understood that these embodiments are merely illustrative of the principles and applications of the present disclosure. It is therefore to be further understood that numerous modifications may be made to the illustrative embodiments and that other arrangements and patterns may be devised without departing from the spirit and scope of the embodiments according to the disclosure. Furthermore, particular features, structures, materials, or characteristics may be combined in any suitable manner in any one or more of the embodiments.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. A collapsible container for a fluid, comprising:
a flexible material, defining an internal working volume;
at least one collapsible baffle adhered within the working volume of said collapsible container; said at least one baffle having one or more channels for delivering one or more fluids into the working volume via at least one hole in said one or more channels;
one or more channels in said container for exiting or venting fluids from the working volume; and
an impeller assembly disposed at least partially within said working volume of said container.

2. The container of claim 1, wherein said baffle comprises one of a ladder-shaped baffle or an X-shaped baffle.

3. The container of claim 1, wherein said working volume is a closed volume.

4. The container claim 1, wherein said container is a two-dimensional bag, a three-dimensional bag, or a bioreactor.

5. The container of claim 1, wherein the at least one baffle comprises collapsible pockets for housing a rigid member.

6. The container of claim 1, wherein the at least one baffle is capable of delivering a gas or liquid to said working volume above a surface level of said fluid, at a surface level of said fluid, or below a surface level of said fluid via said holes.

7. The container of claim 1, wherein the at least one baffle is capable of delivering a liquid that includes feed, nutrients, buffer solution, and/or other processing aids.

8. The container of claim 5, wherein the pockets are tapered from a narrowest point adjacent the bottom wall to a widest point adjacent the top wall.

9. The container of claim 1, wherein the one or more channels traverses a bottom portion of the bag and extends to an upper position above a liquid surface level to deliver an antifoam agent onto the liquid surface.

10. The container of claim 1, wherein the one or more channels traverses a bottom of the bag to an upper position above a liquid surface level to deliver gases to the container,

11. The container of claim 1, wherein the one or more channels are formed of flexible plastic tubing for delivery of pressurized gases or liquids.

12. The container of claim 1, wherein the one or more channels extend from a first position adjacent the bottom wall on the one or more sidewalls to a second position on one more of the sidewalls adjacent the top wall at least tangentially opposite that of the first position.

13. The container of claim 1, comprising two or more channels.

14. The container of claim 1, comprising three or more channels.

15. The container of claim 1, comprising four or more channels,

16. The container of claim 1, further comprising a collapsible dip tube.

17. A method of mixing a fluid in a collapsible container, comprising:
providing a container defining a working volume;
providing an impeller assembly mounted at least partially within said working volume of said container;
placing a collapsible baffle having one or more channels, said channels having holes, within said working volume of said container;
introducing fluid to be mixed into said container to a level only partially submerging said baffle; and
driving said impeller assembly to mix said fluid, wherein said baffle minimizes the formation of any vortex during said mixing.

18. The method of claim 17, wherein said container is a bioreactor.

19. (canceled)

20. The method of claim 17. wherein said fluid comprises cells.

21. The method of claim 17, wherein said fluid further comprises microcarriers for said cells.

22. The method of claim 17, wherein the one or more channels extend from a first position adjacent a bottom wall on the one or more sidewalls of the container to a second position on one more sidewalls adjacent a top wall of the container at least tangentially opposite that of the first position.

23. (canceled)

24. The method of claim 17, wherein liquids and/or gases exit the working volume from the one or more channels in said container.
